(19) United States
(12) Patent Application Publication
(10) Pub. No.: US 2016/0033480 A1
(43) Pub. Date: Feb. 4, 2016

(54) Immunovir and Components, Immunovir A, B, C, D Utility and Useful Processes

(71) Applicant: Amy Huimeei Lo, Brea, CA (US)

(72) Inventors: Yong Chi Lin, Taipei (TW); Po Wen Lin, Taipei (TW); Po Yu Lin, Taipei (TW); Amy Huimeei Lo, Brea, CA (US); Jing Meei Lin, Taipei (TW)

(21) Appl. No.: 14/107,086

(22) Filed: Dec. 16, 2013

(51) Int. Cl. G01N 33/50; C08B 37/00

(52) U.S. Cl. CPC G01N 33/5047; C08B 37/0057; G01N 33/5055

(57) ABSTRACT

A complete remedy for AIDS is difficult to obtain. As such, a useful process was designed to search for an anti-HIV₁ agent that has an immuno response modification activity capable of releasing immuno suppression, activating killer cells to destroy persistent infection cells, elevating antibody titer to activate ADCC activity, and vice versa.

The process consists of 4 elements: guinea pig or mouse peritoneal derived adherent macrophages/monocytes as effector cells; cyclophosphamide as an immuno suppressor; chicken RBC as target cells; and the anti-HIV₁ agent candidate to be examined.

Immunovir and components were isolated from Pyrus serotina Rehder and other species of Rosaceae by column chromatography.

Another useful process is the comparison of fluorescent antibody titer patterns among one round, two round, and non-medicated infected monkeys.

The results of such processes can show that the anti-HIV₁ agent, such as these immunovirs, are noble candidates for the complete remedy of AIDS.

### STATEMENT REGARDING FEDERALLY SPONSORED RESEARCH OR DEVELOPMENT

N/A

### REFERENCE TO SEQUENTIAL LISTING, A TABLE, OR A COMPUTER PROGRAM LISTING APPENDIX SUBMITTED ON A COMPACT DISC

N/A

### CROSS-REFERENCE TO RELATED APPLICATIONS & CONTINUITY DATA

N/A

### BACKGROUND OF THE INVENTION

AIDS is extremely difficult to cure for many reasons. First, nucleoside analogue reverse transcriptase inhibitor (NART I) and non-NART I or protease inhibitor are competitive inhibitors. They do not inhibit human immunodeficiency virus (HIV) replication completely, and can induce persistent infecting cells, resting cells, and drug fasting easily.

Second, HIV is a highly variable virus. Isolating the virus from different organs of the same patient would not result in identical samples of the virus.

Third, HIV has a lysis effect on CD+ cells. Its constituents, particularly surface antigens, have difficulty signaling Th cells. Thus, an inadequate amount of antibody is produced to activate the antibody-dependent cytotoxic cells (ADCC) to kill the monocytes and macrophages that are persistently infected with the virus.

Therefore, carriers of HIV have consistently low antibody titer, and continuously spread out HIV. Based on the understanding of the HIV mechanism, this agent intends to activate killer cells, which can damage cells persistently infected with HIV. By releasing immature and non-infections virion and surface antigens to promote antibody production, ADCC activity can be activated (and vice versa) to ultimately cure AIDS.

### FIELD OF THE INVENTION

This present invention relates to the field of anti-HIV agents with good curative effects against AIDS.

### SUMMARY OF THE INVENTION

This invention is a low-cost anti-HIV agent with good curative effects against AIDS. Plant ingredients of this agent were obtained via plant harvest, ingredient extraction, refinement, and specification. Such ingredients were used in anti-HIV in vitro tests, anti-AIDS in vivo tests, and adverse effect and safety tests.

The agent has been proven to inhibit HIV replication in vitro and cure SIVmac L28 infection in vivo. It provides good curative effects against AIDS with low adverse effects and is a safe and low-cost anti-HIV and anti-AIDS agent.

### BRIEF DESCRIPTION OF THE DRAWINGS/FIGURES

FIGS. 1-1 and 1-2 show the electrophoresis patterns of several immunovir samples.

FIGS. 2-1 to 2-4 show the analytical chemical results of Immunovir A

FIGS. 3-1 to 3-2 show the analytical chemical results of Immunovir A-HCl hydrolysate

FIG. 4-1 shows the response of target chicken red blood cells (cRBC) and guinea pig peritoneal derived adherent macrophages culture with R7½G as effector cells (gMφ) to Immunovir A(1) and/or cyclophosphamide

FIG. 4-2 shows a graphical representation of the experimental system

FIG. 4-3 shows the effects of immunovir and/or cyclophosphamide on gMφ activity in vitro

FIG. 5 shows immunovir A, B, C, and D activation of Mφ/Mo in mice

FIG. 6 shows the anti-HIV₁ activities of immunovir and AZT in vitro

FIG. 7 shows the therapeutic efficacy of immunovir against M. cyclopis infected with SIVmac

Table 1 summarizes the NMR data of Immunovirs A, B, C, D and xylan

Table 2 summarizes the proton and 13-C NMR results on Immunovir A(1)

Table 3 summarizes the effects of Immunovir on killer cell activity in mice

Table 4 summarizes the effect of Immunovir and Cyclophosphamide on serum hemolysin titer in cRBC immunized mice

Table 5 summarizes the hemagglutination activity of immunovirs

Table 6 summarizes the inhibition of reverse transcriptase activity

### DETAILED DESCRIPTION OF THE INVENTION

The original plant material for this invention comes from genus Rosaceae, family Pyrus, i.e., P. lindley Rehder, P. serotina Rehder, P. pyriofolia Nak, which are spread around the world and easily obtained.

The constituents of the plant are extracted, isolated, purified, and analyzed for its physical-chemical characteristics, to get anti-HIV and/or anti-AIDS agents through in vitro and in vivo testing.

### EXAMPLES

### Example 1

Exactly 36 kg of dried bark was ground up into powder and adequately soaked with 200 liters of 10-50% v/v ethanol in water. Despite the bark, the extract from the root and stem could also be evaporated by reducing pressure at 55° C. to get concentrate and, if necessary, lyophilisation.

The bioactivity of the lyophilized-powder concentrate, i.e., crude extract of target constituents, will remain active for several years.

### Example 2

To isolate bioactive immunovir, 100 mL of crude extract or lyophilized-powder concentrate was loaded into a column (90 mmu×760 mmL) in order to isolate immunovir by cellulose adsorption chromatography using a menstrum of water.

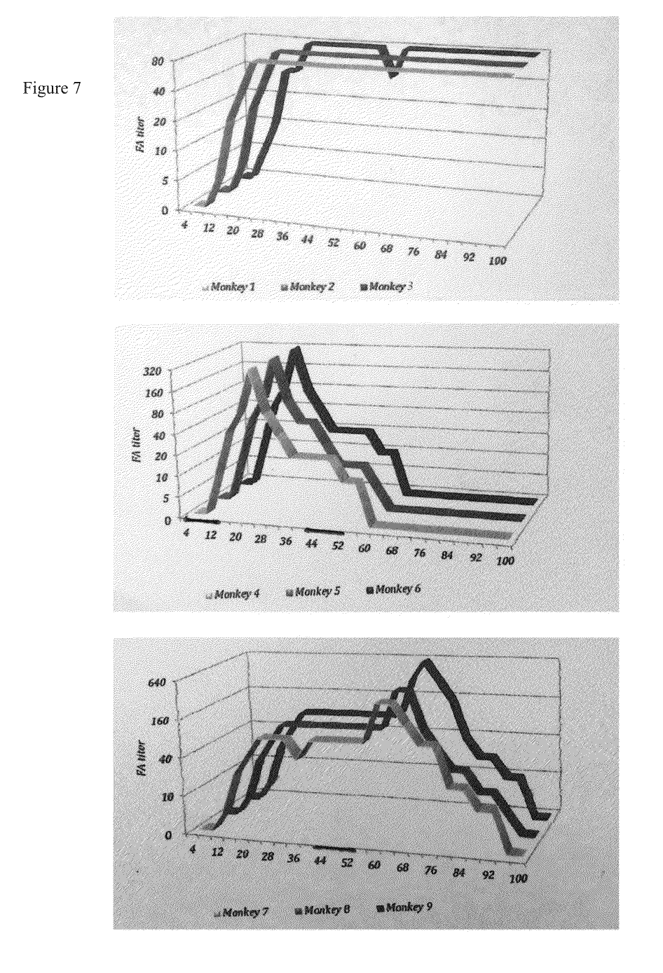

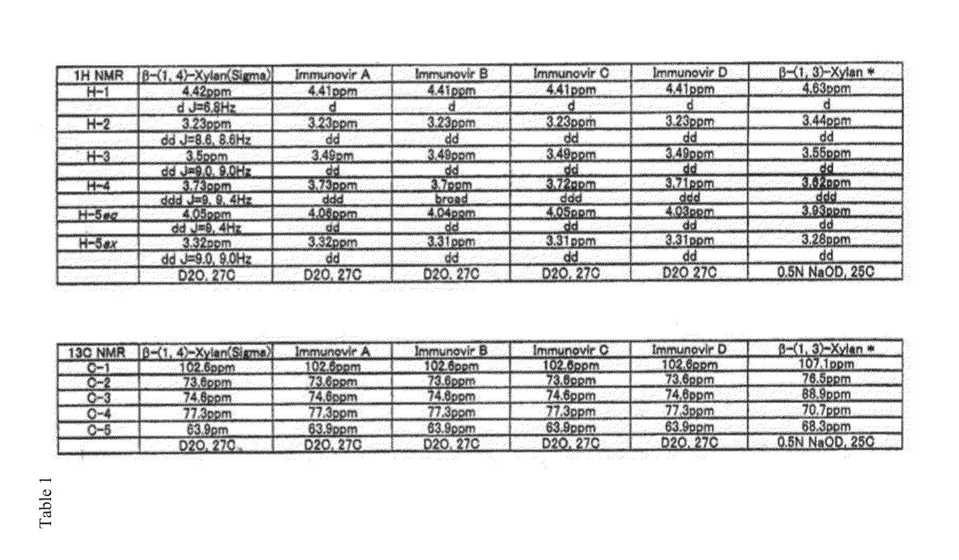

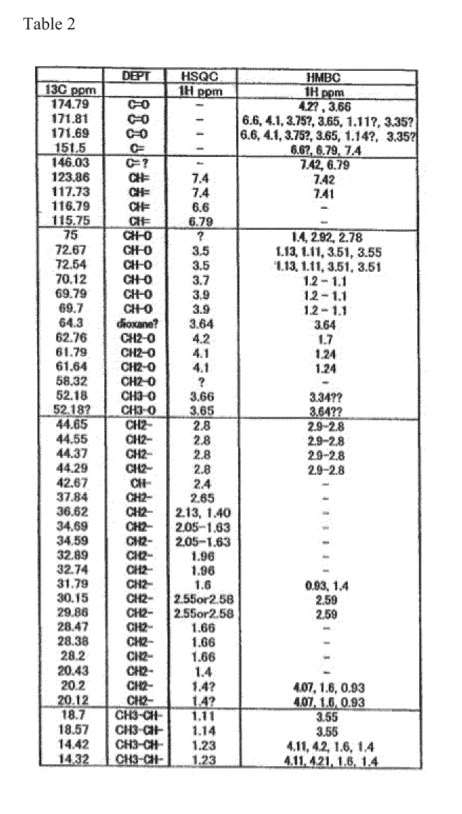

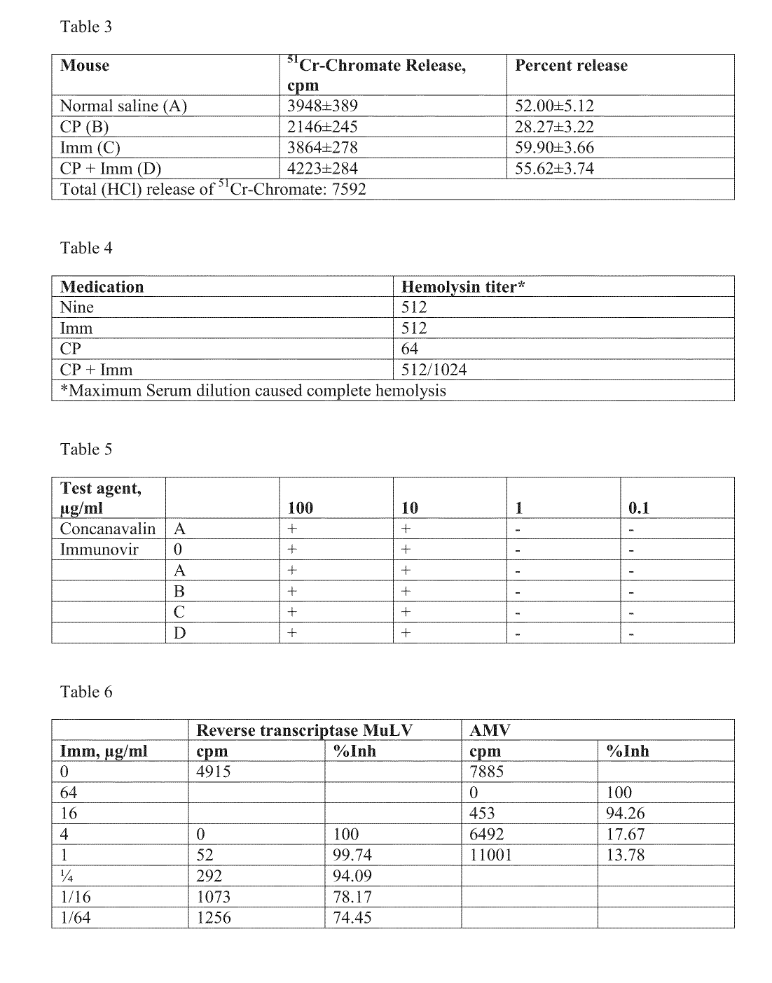

Bioactive reddish purple fraction was collected from the column, concentrated under reduced pressure, and lyophilized to give 539 g of immunovirs. Through repeated cellulose column chromatography, the immunovir solution could be separated into four components, i.e., immunovirs A, B, C, and D. Each of the immunovirs showed equivalent biological activity (FIG. 7, Table 5).

### Example 3

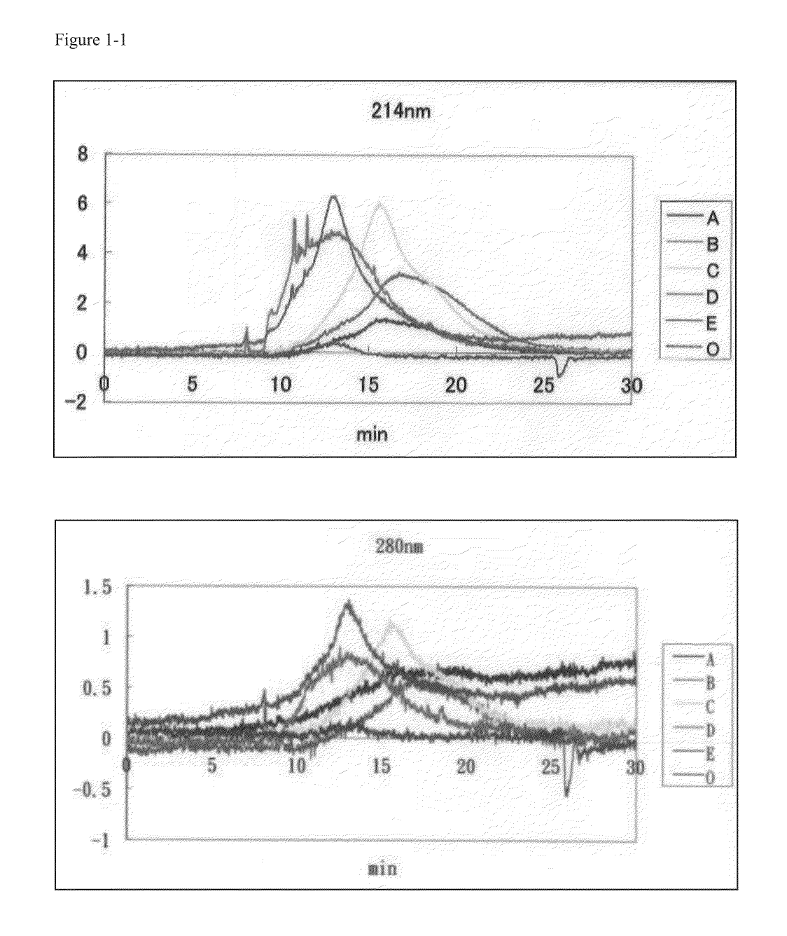

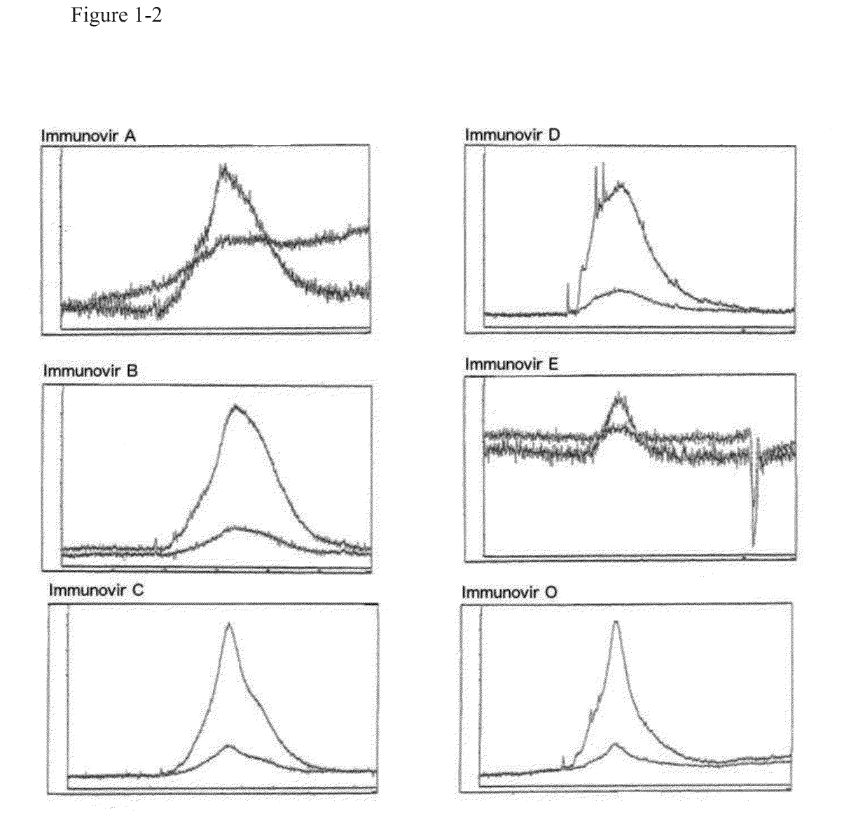

As shown in FIG. 1, immunovirs were separated into categories of A, B, C, D, and E by the differences of location and absorption between peaks in the capillary electrophoresis fingerprint. Through the use of free flow electrophoresis, immunovir separation could be performed continuously and inexpensively. Among these, immunovir E was found to have the same low bioactivity as methyl-a-mannopyranoside and thus, should be removed from this invention.

### Example 4

Characterization by NMR and mass spectra (FIGS. 2-1, 2-2, and 2-3) show the 1H and 13C NMR spectra of immunovir A(1). The 1H-NMR spectrum of 1 in D₂O indicated five methane protons connected to an oxygen atom at 4041 ppm (H-1), 3.23 ppm (H-2), 3.5 ppm (H-3), 3.73 ppm (H-4), and one methylene at 3.32 ppm (H-5ax), 4.06 ppm (H-5eq), respectively. Other signals with relatively low intensity were found in the region of 5.2 to 3.1 ppm. The observed coupling constants of the signals were as follows: H-1(d) J_{1,2}=7 Hz, H-2(d,d) J2,3=9 Hz, H-3(d,d) J3,4=9 Hz, H-4(d,d,d) J4,5eq=4 Hz, respectively. These large coupling constants indicated all axial protons in hexose ring.

The 13C-NMR spectrum of 1 showed five carbon signals: four methane signals at 102.6 ppm (C-1), 73.6 ppm (C-2), 74.6 ppm (C-3), 77.3 ppm (C-4), and one methylene at 63.9 ppm (C-5), respectively. The above described results suggested the presence of xylose in 1. The 1H and 13C NMR spectra of acid hydrolysate of 1 with 3N HCl are shown in FIGS. 3-1 and 3-2. The main hydrolysis product was a mixture of a and B xylose.

On the anomeric configuration of xylose moiety in 1, the observed large coupling constant (J1,2=7 Hz) of H-1 and the chemical shift at 102.6 ppm of C-1 clearly indicate B configuration.

The 13C chemical shift of C-4 in 1 was observed at 77.3 ppm due down field shift by glycosidic linkage, and it corresponded to that of beech wood B-1,4-xylan purchased from Sigma. The C-3 signal in B-1,3-xylan from Caulerpa brachypus (Tohru Yamagaki, et al., Biosci. Bioch. Bioche. 60(8), 1222-1228, 1996) was observed at 88.9 ppm. Therefore, 1 has a linear repeating structure unit that is related to beech wood B-1,4-xylan, and partial chemical structure of 1 would be less-branded polysaccharide.

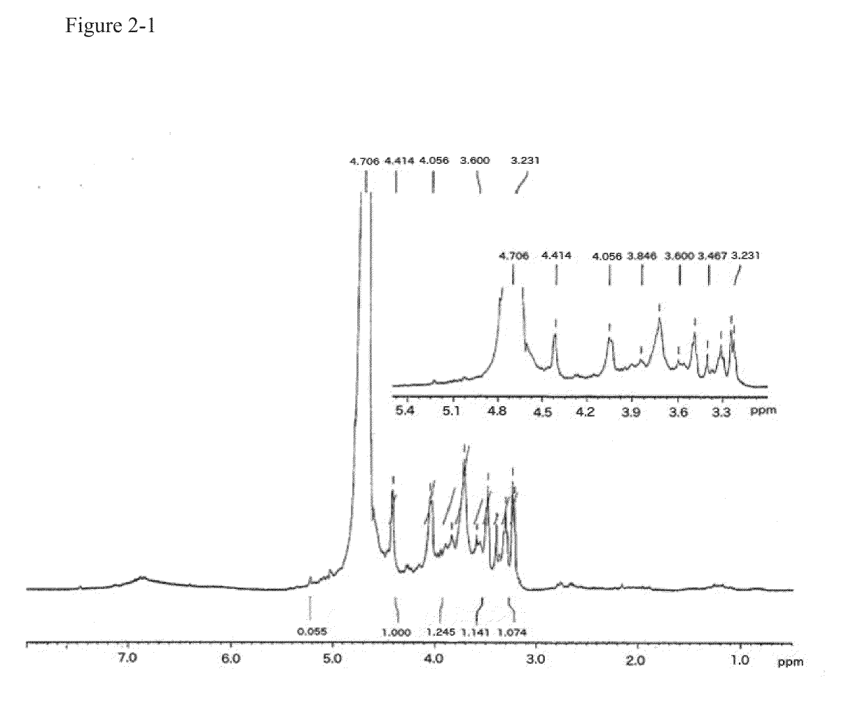

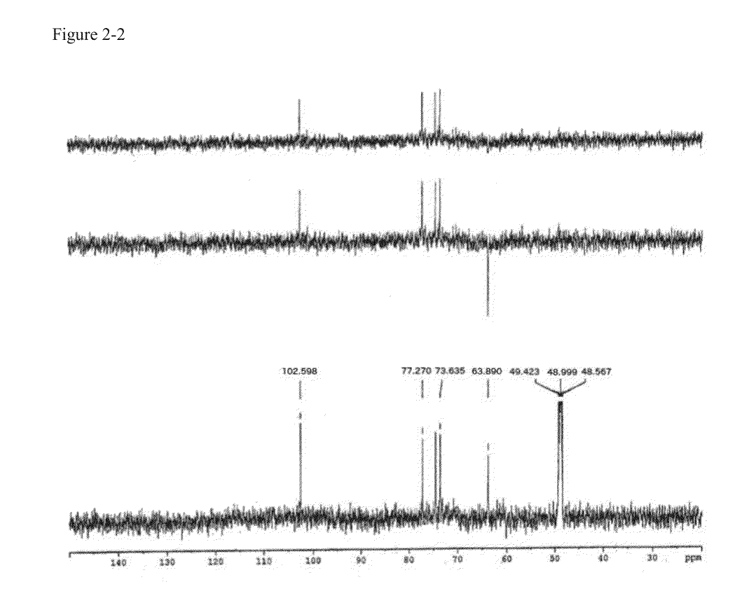

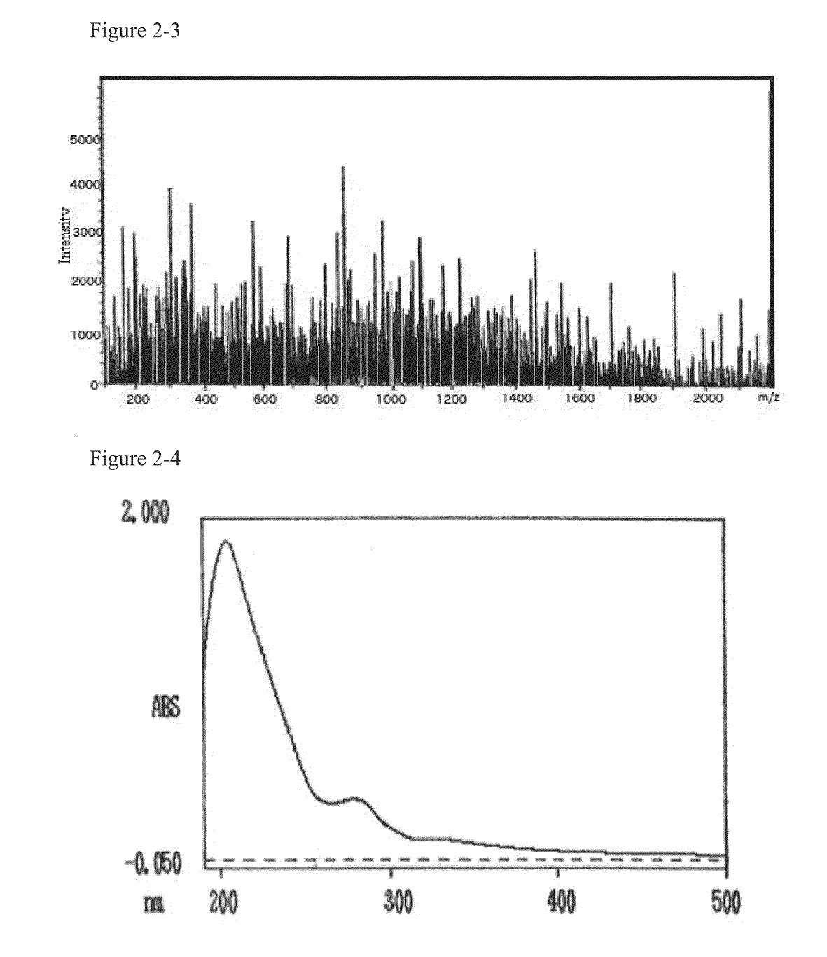

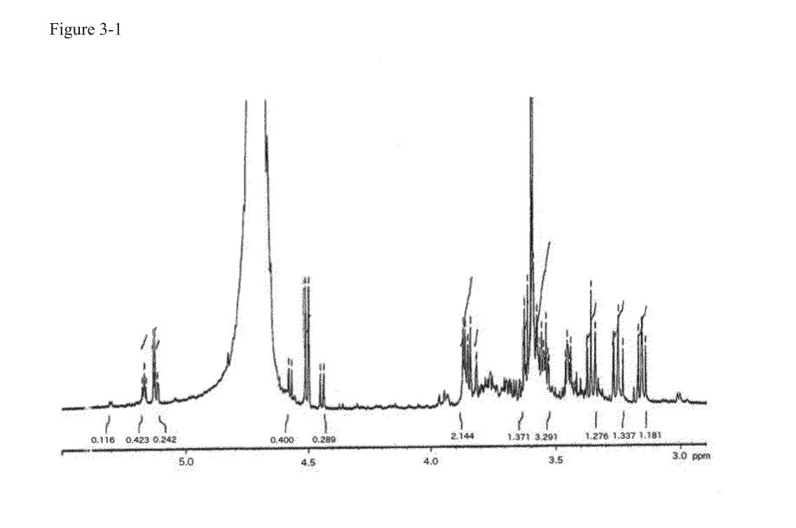

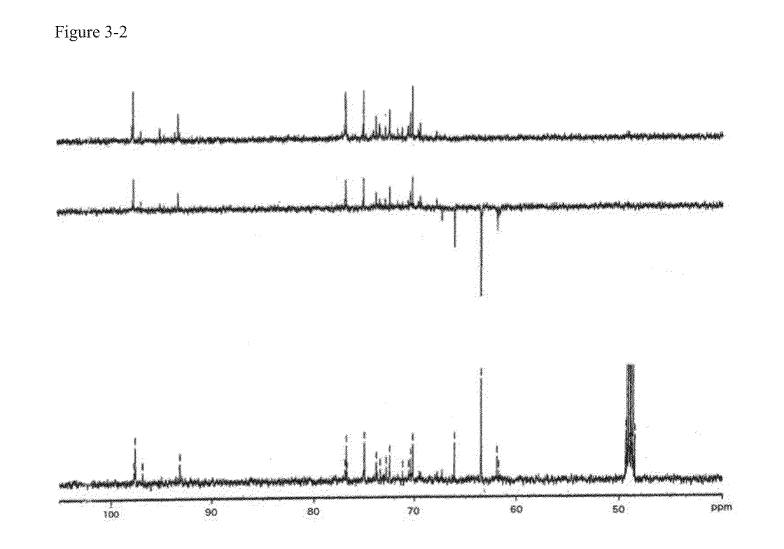

As shown in FIG. 2-3, the molecular weight of immunovir A was immeasurable as detection went beyond the measuring extent of ESI mass spectrometer. It should be more than 10,000 because immunovir A did not pass through the 10 kDA permeable membrane.

Table 1 displays brief specifications of immunovirs. The UV spectrum of immunovir A (FIG. 2-4) indicated the presence of a chromophore in the molecule. The chromophores released from skeletal moiety is very unstable. One of the chromophores, immunovir chrom A(1), isolated from 3N-HCl hydrolysate of immunovir A by/hatman 3MM paper chromatography with upper layer of n-butanol:ethanol:H2O::3:1:3 as menstrum, 1H, 13C, HSQC, and HMBC NMR spectra analysis was presented in the summary of 1H-13C correlation experiment (Table 2) indicated that signals of the chromophor is similar to coumarin, except for the lack of a signal representing that of a carbonyl group at 160 ppm was not observed. Abundant —CH2 signals also indicated presence of a fatty acid moiety.

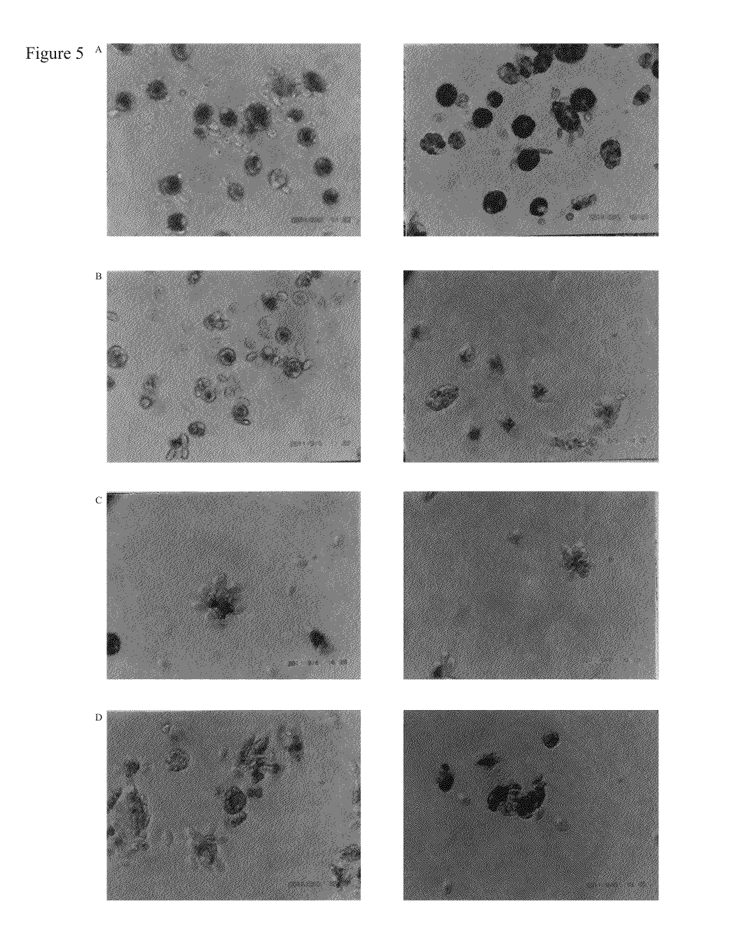

According to the data on Table 1 and FIG. 5, immunovirs A, B, C, and D have similar polysaccharide moieties, physiochemical properties and biological activities. They would be merely different from each other in the number of repeating unit and the position of branch as well as minor saccharide and chromophore moiety.

### Example 5

Effect on cellular immunity in vitro:

The effector (killer) cells for this experiment were adherent macrophages (gMφ) derived from guinea pig's peritoneal fluid cultured with RTAG (RPMI 1640 enriched with 7.5% v/v guinea pig serum). Cyclophosphamide (CP) was used as an immunosuppressive agent. Chicken red blood cells (cRBC) as target cells.

The experiment consisted of four series of experiments: gMφ+cRBC, gMφ+Imm+cRBC, gMφ+CP+cRBC, and gMφ+CP+Imm+cRBC, and immunovir (Imm) as immune response modifier.

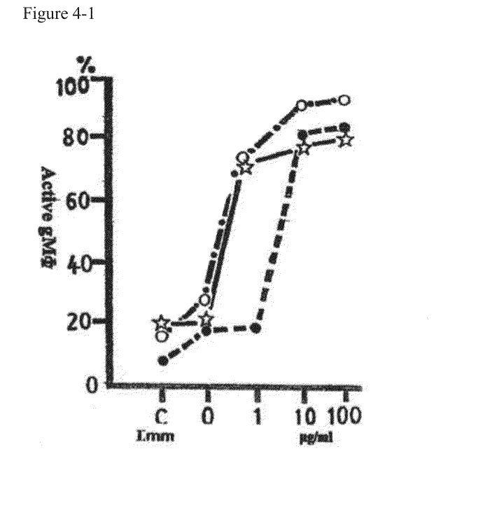

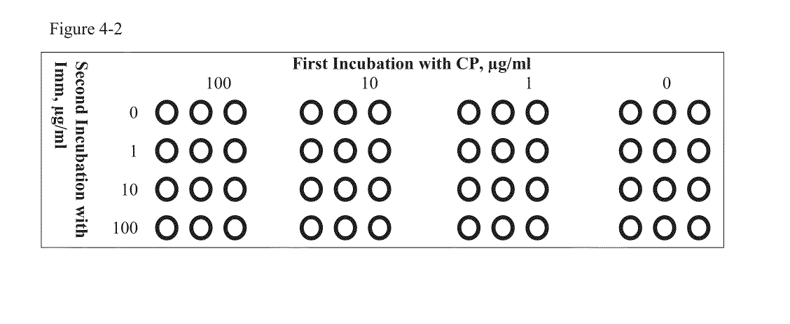

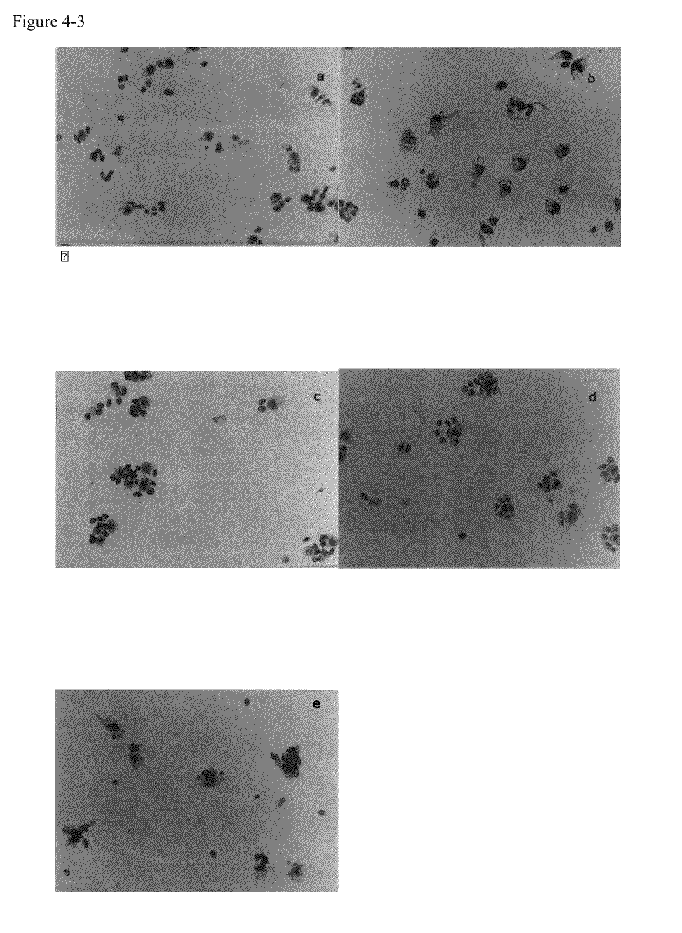

A 200 to 250 g guinea pig was injected with 1 mL of thioglycolate medium, after 20 hours adherent macrophages derived from abdominal cavity (gMφ) were collected by aid of RPMI 1640, suspended in RTAG, then 0.9 mL of solution was pipetted into thirteen Falcon 12-well culture plates. The plates for group 1 and 2 to group 4 were 1 and 3, respectively. See FIG. 4-2. Cyclophosphamide (CP) was added into the wells from group 2 to group 4 so that the final concentration was 1, 10, and 100 ug/mL respectively. In other words, each well was filled with 1 ug/mL CP in group 2, 10 ug/mL in group 3, and 100 ug/mL in group 4. After incubation in a 5% CO₂ incubator (35-37° C.), the medium in each well was replaced with fresh R7½G from group 1 (only 1 plate for control) to group 4. Then Imm was added from lane 1 to lane 4 (3 wells per lane) that the final concentration were 0, 0.1, 1, and 10 ug/mL, respectively. That was to say, from group 2 to group 4, lane 1 was a CP control, and lane 1 in plate 1 was gMφ control. After subsequent incubation in 5% CO₂ incubator, medium in each well of plate was replaced with 0.2% cRBC-containing RPMO 1640. cRBCs were sucked out after another two to six hours of incubation and adhered cells (gMφ and attached cRBC) were stained by Liu Stain. The percentage of active gMφ in 300 gMφ or above was calculated under the optical microscope, and the results were shown in FIGS. 4-1, as C and O in the figure indicated that activity of gMφ were unrecoverable with the removal of CP suppression in spite of the second 20-hours incubation, and demonstrated that a 1/10 concentration of immunovir could remove the suppression of CP.

FIG. 4-3 demonstrated that immunovir removed the immuno suppression of CP and increased the killer cell activity of individual gMφ.

FIG. 4-1 also demonstrated that immunovir neutralized 10-fold immuno suppression of CP.

### Example 6

Effect of mice macrophages/mononuclear cells activities in vivo.

Mice were injected with cyclophosphamide 200 mg/kg.b.w and 100 mg/kg.b.w via tail vein in the morning of day 1 and day 2, respectively. Two mice of each group were injected with 10 mg/kg.b.w of immunovir (O, mixture), immunovir A, B, C, D, or 20 mg/kg.b.w of AZT via tail vein in the afternoon from day 2 to day 5, respectively. Each mouse's abdominal cavity was injected with 0.5 mL of RTAC in the afternoon of day 5, and mMφ/Mo were collected from each mouse's abdominal cavity with 10 mL of R7 ½C in the afternoon of day 6. Basal medium rich in deposit cells were taken, and 0.40 mL was pipetted into two wells of flat-bottomed 24-well Falcon culture dish. After incubation with 5% CO₂ for 6 hours, 0.10 mL of cRBC (1%) was added into each well and incubation was carried out for 6 hours or overnight. Then suspended cells, i.e., cRBC, were sucked out, the well was gently washed with 0.5 mL of RPMI 1640, and 0.40 mL of RBMI 1640 and 5 ul of Liu Stain B solution were added again to stain mMφ, whereas cRBCs were unstainable. CP immuno suppression was removed by Immunovir(mix), Immunovir A, B, C, D, etc. but not AZT in mouse's body, and the efficacy of Mφ/Mo activity increased as shown in FIG. 5.

### Example 7

The efficacy of immunovir to mononuclear killer cell activity derived from mouse's spleen:

Twenty male BALB/c mice aged 8 weeks were divided into group A, B, C, and D. Mice in group A were injected with 0.20 mL of normal saline intravenously. Group B received cyclophosphamide (CP) 200 mg/kg.b.w and 100 mg/kg.b.w at day 1 and day 2, and subsequently, received normal saline every day. Group C received immunovir 10 mg/kg.b.w every day. Group D received CP as group B and immunovir as Group C. All mice's spleens were excised at day 7 and spleen-derived mononuclear cells were isolated by Ficol-paque centrifugation.

Yac-1 cells (2×10⁶/mL) were labeled with R20C containing 1 uc/mL of ⁵¹Cr-chromate for 60 minutes at 37° C.

Mouse's spleen-derived mononuclear cells (killer cells) (3×10⁶) and ⁵¹Cr-chromate-labeled Yac-1 cells (6×10⁶) were suspended altogether in 1.0 mL of R20C medium and incubated at 37° C. for 150 minutes, centrifuged with 250 g for 10 minutes, then 0.50 mL of supernatant was taken and dried in the bottle.

Radioactivity was measured by liquid scintillation and toluene-PPO-POPOP was used as a scintillant.

The results were shown in Table 3, the radioactivity released was inhibited by CP, but neutralized by Imm later.

### Example 8

Hemagglutination activity:

Immunovir A, B, C, and D, or concanavalin A with different concentrations were added into 2 mL of normal saline 0.4% v/v cRBC suspension in Kahn tube. The solution was mixed thoroughly and kept at room temperature for 2 hours to observe hema-agglutination. The results were shown in the Table 6. The tested drugs had same activities, and blood cells were firmly agglutinated with concanavalin A and could not be resuspended by shaking, whereas cRBCs agglutinated with immunovir A, B, C, D could be re-suspended evenly by shaking and re-agglutinated. It suggested that the administration of immunovir A, B, C, and D might be injected intravenously without forming a blood clot.

### Example 9

Immunovir enhancement of humoral immunity:

Sixteen normal and CP-immunosuppressing mice were used. Four mice of each group was tested with the effect of immunovir (mix) for the ability of hemolysin (antibody) production of cRBC (antigen) after immunization.

At day 1, mice were injected with 0.05 mL of normal saline (NS) suspension of 0.4% v/v cRBC via tail vein.

Group 1 mice were injected with 0.05 mL NS from day 1 to day 5 (normal control)

Group 2 mice were injected with 0.02 mg of immunovir (10 mg/kg b.w,i.v.).

Group 3 mice were injected with 4.0 mg of cyclophamide (200 mg/kg b.w., i.v.) at day 1, 2.0 mg of CP (100 mg/kg b.w.i.v.) at day 2, and N.S. from day 3 to day 5 (Immunoresponse suppression group).

Group 4 mice were injected with CP at day 1 and day 2 as well as group 3, and 0.20 mg of immunovir (Imm) (10 mg/kg b.w, i.v.) from day 1 to day 5 for the efficacy test of Imm to serum hemolysin or antibody titer.

All mice were sacrificed at day 6, and sera were taken for the test on hemolysin (anti-cRBC antibody) titter.

For a flat-bottomed 96-well (8×12 wells) Falcon plate, 100 uL of mice serum (1:8 dilution) were added into the well 1 in lane 1, then a two-fold dilution series was performed down to the well 12. Besides 60 uL of a 0.25% cRBC, and 50 uL normal guinea pig (GP) serum (1:8 dilution) was added into each well as a complement, and the final concentration was 1 unit/mL.

After incubation in 5% CO₂ incubator at 37° C. for 6 hours, hemolysis was observed under the optical microscope. The hemolysin (antibody) titer was defined as the highest dilution of mice serum with complete hemolysis, and the results were shown in Table 4.

### Example 10

Immunovir Inhibition of reverse transcriptase:

50 uL of reaction solution consisted of 50 mM Tris-HCl (pH 8.3), 10 mM MgCl₂, 2 mM dithiothreitol, 0.1 unit poly A-γ-oligo-dT, 5-unit reverse transcriptase, 60 ug bovine serum albumin, 0.5 mM/uc3H-dTTP, and Imm with different concentration. After incubation at 37° C. for 1 hours, 25 uL of reaction solution were dripped into a Whatman GF/C glass microfiber filter disc. Unreactive ³H-dTTP was washed out with 5% TCA-0.01M pyrophosphate, and the radioactivity was measured by liquid scintillation. The results minus unspecific count (MuLV 85 cpm, AMV 115 cpm) were shown in Table 6.

The data demonstrated that there was a great divergence between immunovirs in the inhibition of reverse transcriptase from different origins.

### Example 11

Anti-HIV₁ activity:

Mononuclear cells derived from human venous blood (1×10⁷ cells/0.80 mL) were suspended in capped Greiner incubation tube with R20C containing 40 ug/mL rIL-2, and 0.10 mL of immunovir (40 ug/mL or diluted with 4 fold) or, as a comparison, AZT (100, 10, or 1 ug/mL), then incubated in 5% CO₂ incubator at 36-37° C. for 21 days.

Cells were precipitated by centrifugation (250 g, 5 minutes) every 3 days, and culture medium were replaced to fresh ones with identical ingredients. For antigen expression, cells were stained by mouse anti-HIV₁P₂₄ IgG₁ and rabbit anti-mouse IgG₁-FITC.

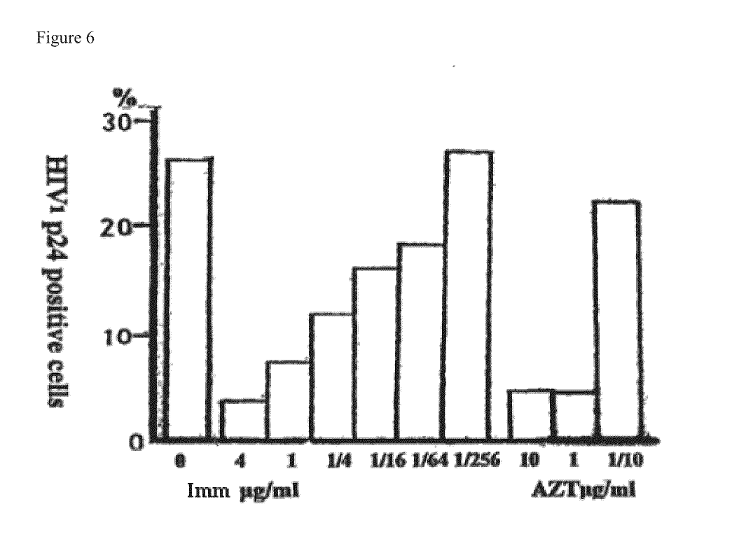

The percentage of cells with HIV₁P₂₄ gag gene expression, i.e., the ratio of fluorescent cells detected by indirect fluorescent antibody technique was calculated. The comparison of anti-HIV₁ activity between immunovir and AZT was shown in FIG. 6.

The anti-HIV₁ activities of immunovir and AZT were approximately the same.

### Example 12

Experimental therapy on SIVmac L28 infection monkeys:

Nine male Macaca cyclopis were divided into 3 groups. Infection and experimental therapy were performed in 3 monkeys of each group. Monkeys were fed with monkey chews and sweet potato twice a day.

Virus suspension for inoculation:

For prevention of non-specific immune response, R20M (RPMI 1640 containing 20% monkey serum) containing 40 u/mL γ 4-IL-2 (recombinant IL-2) and 5 ug/mL PHA-P was used to culture mononuclear cells derived from monkeys' venous blood (mPBMC) and cultured. After SIVmac L28 inoculation, mPBMC (1×10⁶ cells/mL) were incubated in 5% CO₂ incubator at 36-37° C. for 21 days. Culture medium was replaced to fresh one every 3 days.

mPBMC were collected by centrifugation, washed with RPMI 1640, suspended with small volume of RPMI 1640, damaged by repeated freeze and sawing, and heavy virus suspension supernatant was collected after centrifugation to inoculate Macaca cyclopis intravenously.

Assessment of virus population

Human venous blood-derived adherent mononuclear cells in R20C were inoculated with 0.10 mL of 10-fold dilution SIVmac L28 then cultured for 21 days, and virus population was measured by indirect FA technique with self-made monkey anti-SIVmac L28 antiserum and mouse anti-monkey IgG₁-FITC. The 0.10 mL of SIVmac L28 virus suspension for animal inoculation was found out to have 1×10⁷ TCID (tissue culture infective dose). Target cells for assessment of anti-SIVmac L28 FA titer of monkey serum:

Human venous blood-derived adherent mononuclear cells were massively cultured with R20C. After the inoculation of SIVmac L28, R20C was replaced to fresh ones every 3 days. The target cells, i.e., virus-bearing Mφ/Mo cells, were incubated with 5% CO₂ at 36-37° C. for 21 days for FA titer determination. Mφ/Mo cells were dissociated by soaking culture flasks into ice water and collected by centrifugation. Cells were washed by PBS (pH 7.4) for three times, suspended in PBS as Mo/Mo-concentrated suspension. Cell suspension was spread on many 8-well FA slides, the slides were air dried at room temperature, fixed by cold acetone for 10 minutes, air dried again, stored at −20° C. refrigerator until use.

Assessment of monkey serum FA titer:

Target cells were flooded by monkey serum which was given for testing and serial-diluted with PBS. After standing at 37° C. for 30 minutes, serum diluent solution was removed, and slides were placed into PBS and gently stirred for 10 minutes three times. Then, air-dried target cells were flooded by mouse anti-monkey IgGi-FITC with appropriate concentration, counter stained at 37° C. for 30 minutes, washed with PBS three times for the removed of counter stain solution, air dried, flooded by fluorescein-free glycerol and covered by cover glass, and observed under the ultraviolet microscope for the presence of target cells with white-bright fluorescence.

The FA titer of the serum was defined as the highest dilution of monkey serum which was given for testing that produced fluorescent cells.

Experimental therapy on M. cyclopis inoculated with SIVmac L28:

Nine male Macaca cyclopis weighted 4-5 kg were divided into 3 groups, 3 monkeys a group. Three days after intravenous inoculation with 2.0 mL of virus inoculum, group 1 (M4, M5, M6) started first round of immunovir (mixture) (5 mg/kg b.w.i.v.) therapy. The strategy was 1 dose per day, 6 days per week, and followed by 1 day withdrawal for 12 weeks. Forty weeks after virus, immunovir was also administered for 12 weeks for the second round medication. Forty weeks after inoculation, group 2 (M7, M8, M9) started first round of therapy for 12 weeks also. Group 3 (M1, M2, M3), a non-medicated control group, was administered with 5% glucose, in the mean-time other macaques were medicated.

Immunovir efficacy on experimental therapy:

After all Macaca cyclopis were inoculated by SIVmac L28, 2 mL of blood was drawn every two weeks for the assessment of serum FA titer and the assessment was performed for 100 weeks, the results were shown as FIG. 7. Twelve weeks after inoculation of SIVmac L28, monkeys of group 3 were found to have fluorescent antibodies, and after 24 weeks, the titer reached up to a constant value of 80 and persisted for more than 100 weeks. The pattern was identical to human HIV₁ infection. Group 1 (two-round immunovir therapies) were also found to have FA at week 12, and reached a peak value of 160 to 320 at week 24, then gradually decreased. The antibodies of group 1 monkeys vanished after weeks 52 and did not reappear in response to the second round of immunovir medication from week 40 to 52. It was indicated that there was no SIVmac L28 in the monkey's body.

Monkeys of group 2 received one round of immunovir therapy at week 40 after virus inoculation, i.e., the monkeys had become carriers. An increase in FA titer at week 60 was detected, and reached a peak value of 320 to 640 at week 64, then gradually decreased and vanished at week 96. The comparison of pattern for monkey serum's FA titer between groups indicated that immunovir would cure SIVmac L28-infected Macaca cyclopis and might also cure AIDS in humans.

### Recovery of SIVmac L28

At week 100, 2 mL of blood was drawn from every monkey and added with heparin (5 u/m) for standing. Adherent hPBMC-derived Mφ/Mo was infected by mPBMC-rich plasma, and virus could be retrieved from monkeys of group 3 (M1, M2, M3) rather than group 1 (M4, M5, M6) and group 2 (M7, M8, M9), as noted here.

What is claimed is:

1. (canceled)

2. (canceled)

3. (canceled)

4. (canceled)

5. (canceled)

6. (canceled)

7. (canceled)

8. (canceled)

9. (canceled)

10. The purified compound isolated from a process comprising of grounding up dried bark of plants from the genus Rosacea; soaking in 10% to 50% ethanol in water; purifying through the use of cellulose column chromatography; and separating into components through free-flow electrophoresis.

11. The process for screening immune-response modifiers for possible HIV remedy comprised of an immuno suppressor, effector cells, target cells, and the target candidate.

12. The process of claim 11, whereas the immune suppressor is cyclophosphamide.

13. The process of claim 11, whereas the effector cells is either guinea pig or mouse peritoneal-derived macrophages/monocyte.

14. The process of claim 111, whereas the target cell is chicken red blood cells.

15. The process of determining efficacy of a complete remedy of AIDS through animal model experimental therapy, comprising of “first round” medication, “second round” medication, and comparing the antibody titer patterns.
